(19) United States
(12) Patent Application Publication
(10) Pub. No.: US 2026/0283568 A1
(43) Pub. Date: Sep. 24, 2026

(54) SYSTEMS AND METHODS FOR REGISTERING RADIONUCLIDE INFORMATION

(71) Applicant: SHANGHAI UNITED IMAGING HEALTHCARE CO., LTD., Shanghai (CN)

(72) Inventor: Dan WANG, Shanghai (CN)

(73) Assignee: SHANGHAI UNITED IMAGING HEALTHCARE CO., LTD., Shanghai (CN)

(21) Appl. No.: 19/435,662

(22) Filed: Dec. 29, 2025

(30) Foreign Application Priority Data
Dec. 27, 2024 (CN) 202411959997.3

(51) Int. Cl. A61B 6/03; A61B 6/00

(52) U.S. Cl. CPC A61B 6/037; A61B 6/5211; A61B 6/54

(57) ABSTRACT

A method for registering radionuclide information may be provided. The method may comprise obtaining a registration instruction for registering a target radionuclide into a radionuclide library of an emission computer tomography (ECT) scanner, the registration instruction including basic information of the target radionuclide. The method may also comprise, in response to the registration instruction, obtaining photon energy information of the target radionuclide by controlling the ECT scanner to perform an ECT scan on a subject including the target radionuclide. The method may also comprise determining, based on the photon energy information, target values of one or more spectral feature parameters of the target radionuclide. The method may further comprise registering the basic information and the target values of the one or more spectral feature parameters of the target radionuclide into the radionuclide library of the ECT scanner.

### CROSS-REFERENCE TO RELATED APPLICATIONS

This application claims priority of Chinese Patent Application No. 202411959997.3 filed on Dec. 27, 2024, the contents of which are incorporated herein by reference.

### TECHNICAL FIELD

The present disclosure relates to the medical imaging field, and in particular, to emission computed tomography (ECT).

### BACKGROUND

ECT (e.g., single photon emission computed tomography (SPECT), positron emission tomography (PET)) is an advanced medical imaging technology that generates functional metabolic images by detecting gamma rays emitted from radiopharmaceuticals introduced into a subject. It plays an indispensable role in disease diagnosis, staging, and treatment evaluation across various fields such as oncology, neurology, and cardiology.

The quality and accuracy of ECT highly depends on scanning and reconstruction parameters configured for specific radionuclides. Different radionuclides possess distinct physical characteristics, such as energy peaks and decay modes. Therefore, equipment manufacturers typically pre-configure a radionuclide library in ECT systems, where commonly used radionuclides and their corresponding spectral feature parameters (e.g., an energy window, a coincidence time window, attenuation correction coefficients, reconstruction algorithm parameters) are stored for clinical users to directly select during ECT scanning.

However, the demand for novel, non-conventional, or specialized radionuclides in clinical research and practice is continuously growing. These emerging radionuclides are often not included in the pre-configured radionuclide library set by manufacturers at the time of equipment installation. Thus, it is desirable to develop accurate and efficient methods and systems for registering radionuclide information to meet the increasingly demanding requirements of precision and personalized clinical applications.

### SUMMARY

According to an aspect of the present disclosure, a method for registering radionuclide information may be provided. The method may be implemented on a computing device having at least one processor and at least one storage device. The method may comprise obtaining a registration instruction for registering a target radionuclide into a radionuclide library of an emission computer tomography (ECT) scanner, the registration instruction including basic information of the target radionuclide. The method may also comprise, in response to the registration instruction, obtaining photon energy information of the target radionuclide by controlling the ECT scanner to perform an ECT scan on a subject including with the target radionuclide. The method may also comprise determining, based on the photon energy information, target values of one or more spectral feature parameters of the target radionuclide. The method may further comprise registering the basic information and the target values of the one or more spectral feature parameters of the target radionuclide into the radionuclide library of the ECT scanner.

In some embodiments, the one or more spectral feature parameters include at least one of an energy peak, an energy window, a scatter window, or a branching ratio of the target radionuclide.

In some embodiments, the registering the basic information and the target values of the one or more spectral feature parameters of the target radionuclide into the radionuclide library of the ECT scanner comprises: presenting an energy spectrum curve obtained the photon energy information and the target values of the one or more spectral feature parameters to a user; and in response to a confirmation instruction of the target values, registering the basic information and the target values of the one or more spectral feature parameters of the target radionuclide into the radionuclide library of the ECT scanner.

In some embodiments, the energy spectrum curve is presented after the ECT scan.

In some embodiments, the energy spectrum curve is presented during the ECT scan.

In some embodiments, the method further comprises: presenting a drug carrier list including candidate drug carriers; obtaining a selection instruction for selecting one or more target drug carriers from the drug carrier list; and registering associated information between the target radionuclide and the one or more target drug carriers into the radionuclide library.

In some embodiments, the obtaining photon energy information of the target radionuclide comprises: (a) controlling the ECT scanner to start the ECT scan on the subject; (b) obtaining current photon energy information collected by the ECT scanner before a current acquisition time; (c) determining whether a termination condition is satisfied based on the current photon energy information; and (d) in response to determining that the termination condition is satisfied, designating the current photon energy information as the photon energy information and controlling the ECT scanner to terminate the ECT scan; or in response to determining that the termination condition is not satisfied, determining a next acquisition time, designating the next acquisition time as the current acquisition time, and repeating steps (b) through (d).

In some embodiments, the current acquisition time and the next acquisition time are determined based on a preset acquisition time sequence including a plurality of acquisition times arranged in ascending order.

In some embodiments, the basic information includes a half-life period or a branching ratio of the target radionuclide, the registration instruction further includes an initial activity of the target radionuclide, and the preset acquisition time sequence is determined based on the initial activity and the half-life period or the branching ratio of the target radionuclide.

In some embodiments, the determining whether a termination condition is satisfied based on the current photon energy information comprises: obtaining previous photon energy information collected by the ECT scanner before a previous acquisition time of the current acquisition time; and determining whether the termination condition is satisfied based on the previous photon energy information and the current photon energy information.

In some embodiments, the determining whether the termination condition is satisfied based on the previous photon energy information and the current photon energy information comprises: determining an error factor relating to one or more key energy peaks based on the current photon energy information; determining a variation factor between the previous photon energy information and the current photon energy information; and determining whether the termination condition is satisfied based on the error factor and the variation factor.

In some embodiments, in response to determining that the termination condition is not satisfied, the next acquisition time is determined based on the error factor and the variation factor.

In some embodiments, the determining whether a termination condition is satisfied based on the current photon energy information comprises: presenting an energy spectrum curve corresponding to the current photon energy information to a user; and in response to a confirmation instruction of the energy spectrum curve, determining that the termination condition is satisfied; or in response to a re-collection instruction, determining that the termination condition is not satisfied.

In some embodiments, the determining, based on the photon energy information, target values of one or more spectral feature parameters of the target radionuclide comprises: determining, based on the photon energy information, a plurality of recommendation sets, each recommendation set including recommended values of the one or more spectral feature parameters; for each recommendation set, reconstructing an ECT image of the subject based on ECT data collected by the ECT scanner in the ECT scan and the recommended values in the recommendation set; presenting the ECT image and the recommended values corresponding to each recommendation set to a user; and in response to a selection instruction for selecting a target recommendation set from the recommendation sets, designating the recommended values in the target recommendation set as the target values of the one or more spectral feature parameters.

In some embodiments, the method further comprises: obtaining a selection instruction for selecting the target radionuclide for a second subject from the radionuclide library; obtaining third photon energy information collected by the ECT scanner in a second ECT scan on the second subject; determining whether the selection instruction is accurate based on the target values of the one or more spectral feature parameters and the third photon energy information; and in response to determining that the selection instruction is not accurate, generating a prompt.

In some embodiments, the method further comprises: uploading the basic information and the target values of the one or more spectral feature parameters of the target radionuclide to a shared database.

According to another aspect of the present disclosure, a method for registering radionuclide information may be provided. The method may be implemented on a computing device having at least one processor and at least one storage device. The method may comprise obtaining a registration instruction for registering a target radionuclide into a radionuclide library of an emission computer tomography (ECT) scanner. The method may comprise, in response to the registration instruction, obtaining photon energy information of the target radionuclide by controlling the ECT scanner to perform an ECT scan on a subject including the target radionuclide. The method may also comprise determining, based on the photon energy information, an energy spectrum curve. The method may further comprise presenting the energy spectrum curve to a user. The method may further comprise determining, based on the energy spectrum curve, target values of one or more spectral feature parameters of the target radionuclide. The method may comprise registering the target values of the one or more spectral feature parameters of the target radionuclide into the radionuclide library of the ECT scanner.

According to still another aspect of the present disclosure, a method for registering radionuclide information may be provided. The method may be implemented on a computing device having at least one processor and at least one storage device. The method may comprise obtaining a registration instruction for registering a target radionuclide into a radionuclide library of an emission computer tomography (ECT) scanner, the registration instruction including basic information of the target radionuclide. The method may comprise, in response to the registration instruction, controlling the ECT scanner to perform an ECT scan on a subject including the target radionuclide, wherein the ECT scan includes a first scan period and a second scan period, and the first scan period is earlier than the second scan period. The method may also comprise: during the first scan period, obtaining photon energy information of the target radionuclide based on scan data collected during the first scan period; determining, based on the photon energy information, target values of one or more spectral feature parameters of the target radionuclide; and registering the basic information and the target values of the one or more spectral feature parameters of the target radionuclide into the radionuclide library of the ECT scanner. The method may further comprise reconstructing an ECT image of the subject based on the target values of one or more spectral feature parameters of the target radionuclide and scan data collected during the first scan period and the second scan period.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary ECT system according to some embodiments of the present disclosure;

FIG. 2 is a schematic diagram illustrating exemplary hardware and/or software components of an exemplary computing device according to some embodiments of the present disclosure;

FIG. 3 is a block diagram illustrating an exemplary processing device according to some embodiments of the present disclosure;

FIG. 4 is a flowchart illustrating an exemplary process for registering radionuclide information according to some embodiments of the present disclosure;

FIG. 5 is a flowchart illustrating an exemplary process for obtaining photon energy information of a target radionuclide according to some embodiments of the present disclosure;

FIG. 6 is a schematic diagram illustrating an exemplary process for determining whether a termination condition is satisfied according to some embodiments of the present disclosure;

FIG. 7 is a schematic diagram illustrating an exemplary interface for energy spectrum information detection according to some embodiments of the present disclosure;

FIG. 8 is a schematic diagram illustrating an exemplary process for confirming values of spectral feature parameters for an ECT scan according to some embodiments of the present disclosure; and

FIG. 9 is a schematic diagram illustrating an exemplary interface for registering radionuclide information according to some embodiments of the present disclosure;

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assemblies of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The terms “pixel” and “voxel” in the present disclosure are used interchangeably to refer to an element of an image.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, a representation of a subject (e.g., an object, a patient, or a portion thereof) in an image may be referred to as “subject” for brevity. For instance, a representation of an organ, tissue (e.g., a heart, a liver, a lung), or an ROI in an image may be referred to as the organ, tissue, or ROI, for brevity. Further, an image including a representation of a subject, or a portion thereof, may be referred to as an image of the subject, or a portion thereof, or an image including the subject, or a portion thereof, for brevity. Still further, an operation performed on a representation of a subject, or a portion thereof, in an image may be referred to as an operation performed on the subject, or a portion thereof, for brevity. For instance, a segmentation of a portion of an image including a representation of an ROI from the image may be referred to as a segmentation of the ROI for brevity.

Conventionally, for a new radionuclide that is not included in a radionuclide library of an ECT scanner, a user is required to manually search through literature or manufacturer documentation to obtain spectral feature parameters of the new radionuclide and input them into the radionuclide library. This approach suffers from low efficiency and a high risk of human error, which may consequently lead to degraded imaging quality and reduced diagnostic accuracy.

To address the above problems, the present disclosure provides systems and methods for registering radionuclide information. The methods may include the following operations. A registration instruction for registering a target radionuclide into a radionuclide library of an ECT scanner may be obtained. The registration instruction includes basic information of the target radionuclide. In response to the registration instruction, photon energy information of the target radionuclide may be obtained by controlling the ECT scanner to perform an ECT scan on a subject including the target radionuclide. Target values of one or more spectral feature parameters of the target radionuclide may be determined based on the photon energy information. The basic information and the target values of the one or more spectral feature parameters of the target radionuclide may be registered into the radionuclide library of the ECT scanner.

Compared with the conventional approaches, the methods and systems of the present disclosure can rapidly determine accurate values of the spectral feature parameters of the target radionuclide. Moreover, the methods and systems of the present disclosure may be automatically implemented with reduced or minimal or without user intervention, which is more efficient (e.g., reducing the workload of a user and the time needed for registering radionuclide information).

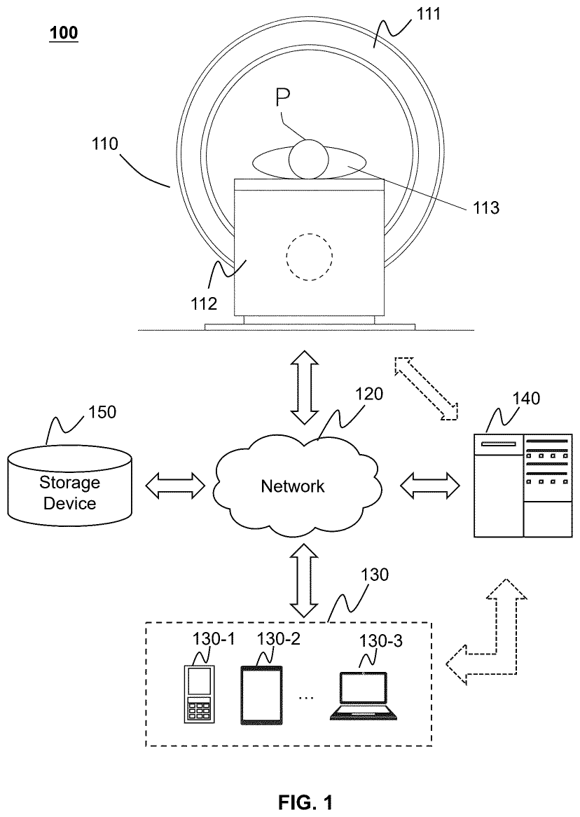

FIG. 1 is a schematic diagram illustrating an exemplary ECT system according to some embodiments of the present disclosure. As illustrated in FIG. 1, the ECT system 100 may include an ECT scanner 110, a network 120, a terminal device 130, a processing device 140, and a storage device 150. The components of the ECT system 100 may be connected in various manners.

The ECT scanner 110 may be configured to acquire scan data (also referred to as ECT data) relating to an object. For example, the ECT scanner 110 may scan the object or a portion thereof that is located within its detection region and generate the scan data relating to the object or the portion thereof.

In some embodiments, the ECT scanner 110 may include a gantry 111 and a couch 112. The gantry 111 may form an accommodation activity for accommodating a subject 113 to be scanned. The couch 112 may be used to support the subject 113. In some embodiments, the ECT scanner 110 may be a single-modality scanner, for example, a SPECT scanner, PET scanner. In some embodiments, the ECT scanner 110 may be a multi-modality scanner, for example, a positron emission tomography-computed tomography (PET-CT) scanner, etc.

The network 120 may facilitate the exchange of information and/or data. For example, the processing device 140 may obtain, via the network 120, scan data relating to the subject 113 or a portion thereof from the ECT scanner 110. In some embodiments, the network 120 may be any type of wired or wireless network, or a combination thereof.

The terminal device 130 may enable interactions between users and components of the ECT system 100. The terminal device 130 may include a mobile device 130-1, a tablet computer 130-2, a laptop computer 130-3, or the like, or any combination thereof. In some embodiments, the terminal device 130 may be part of the processing device 140. In some embodiments, the terminal device 130 may be omitted.

The processing device 140 may process data relating to the ECT system 100. In some embodiments, the processing device 140 (e.g., one or more modules illustrated in FIG. 3) may perform the methods of the present disclosure. For example, any one of processes 400-500 may be implemented as a set of instructions (e.g., an application) stored in a storage device (e.g., the storage device 150). The processing device 140 may execute the set of instructions and may accordingly be directed to perform the processes 400-500. Merely by way of example, the processing device 140 may obtain a registration instruction for registering a target radionuclide into a radionuclide library of the ECT scanner 110, and register radionuclide information of the target radionuclide by performing the process 400.

In some embodiments, the processing device 140 may be a single server or a server group. The server group may be centralized or distributed. In some embodiments, the processing device 140 may be local or remote. Merely for illustration, only one processing device 140 is described in the ECT system 100. However, it should be noted that the ECT system 100 in the present disclosure may also include multiple processing devices. Thus, operations and/or method steps that are performed by one processing device 140 as described in the present disclosure may also be jointly or separately performed by multiple processing devices. For example, if in the present disclosure the processing device 140 of the ECT system 100 executes both process A and process B, it should be understood that the process A and the process B may also be performed by two or more different processing devices jointly or separately in the ECT system 100 (e.g., a first processing device executes process A and a second processing device executes process B, or the first and second processing devices jointly execute processes A and B).

The storage device 150 may store data, instructions, and/or any other information. In some embodiments, the storage device 150 may store data obtained from the processing device 140, the terminal device 130, and/or the ECT scanner 110. For example, the storage device 150 may store scan data collected by the ECT scanner 110. As another example, the storage device 150 may store the radionuclide information of the target radionuclide. In some embodiments, the storage device 150 may store data and/or instructions that the processing device 140 may execute or use to perform exemplary methods described in the present disclosure.

It should be noted that the above description of the ECT system 100 is merely provided for the purposes of illustration, and not intended to limit the scope of the present disclosure. For persons having ordinary skills in the art, multiple variations and modifications may be made under the teachings of the present disclosure. For example, the ECT system 100 may include one or more additional components and/or one or more components of the ECT system 100 described above may be omitted. Additionally or alternatively, two or more components of the ECT system 100 may be integrated into a single component. A component of the ECT system 100 may be implemented on two or more sub-components.

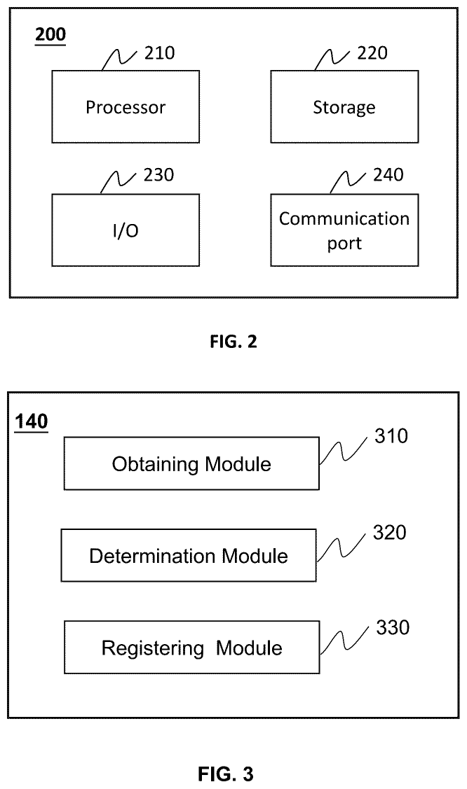

FIG. 2 is a schematic diagram illustrating exemplary hardware and/or software components of an exemplary computing device according to some embodiments of the present disclosure. In some embodiments, the processing device 140 may be implemented on the computing device 200. As illustrated in FIG. 2, the computing device 200 may include a processor 210, a storage 220, an input/output (I/O) 230, and a communication port 240.

The processor 210 may execute computer instructions (program code) and perform functions of the processing device 140 in accordance with techniques described herein. The computer instructions may include routines, programs, objects, components, signals, data structures, procedures, modules, and functions, which perform particular functions described herein. Merely for illustration purposes, only one processor is described in the computing device 200. However, it should be noted that the computing device 200 in the present disclosure may also include multiple processors, and thus operations of a method that are performed by one processor as described in the present disclosure may also be jointly or separately performed by the multiple processors.

The storage 220 may store data/information obtained from the ECT scanner 110, the terminal device 130, the storage device 150, or any other component of the ECT system 100. In some embodiments, the storage 220 may include a mass storage device, a removable storage device, a volatile read-and-write memory, a read-only memory (ROM), or the like, or any combination thereof. In some embodiments, the storage 220 may store one or more programs and/or instructions to perform exemplary methods described in the present disclosure.

The I/O 230 may input or output signals, data, or information. In some embodiments, the I/O 230 may enable user interaction with the processing device 140. In some embodiments, the I/O 230 may include an input device and an output device.

The communication port 240 may be connected to a network (e.g., the network 120) to facilitate data communications. The communication port 240 may establish connections between the processing device 140 and the ECT scanner 110, the terminal device 130, or the storage device 150. The connection may be a wired connection, a wireless connection, or a combination of both that enables data transmission and reception.

It should be noted that the above description of the computing device 200 is merely provided for the purposes of illustration, and not intended to limit the scope of the present disclosure. For persons having ordinary skills in the art, multiple variations and modifications may be made under the teachings of the present disclosure.

FIG. 3 is a block diagram illustrating exemplary processing device 140 according to some embodiments of the present disclosure.

As shown in FIG. 3, the processing device 140 may include an obtaining module 310, a determination module 320, and a registering module 330. As described in FIG. 1, the ECT system 100 in the present disclosure may also include multiple processing devices, and the obtaining module 310, the determination module 320, and the registering module 330 may be components of different processing devices.

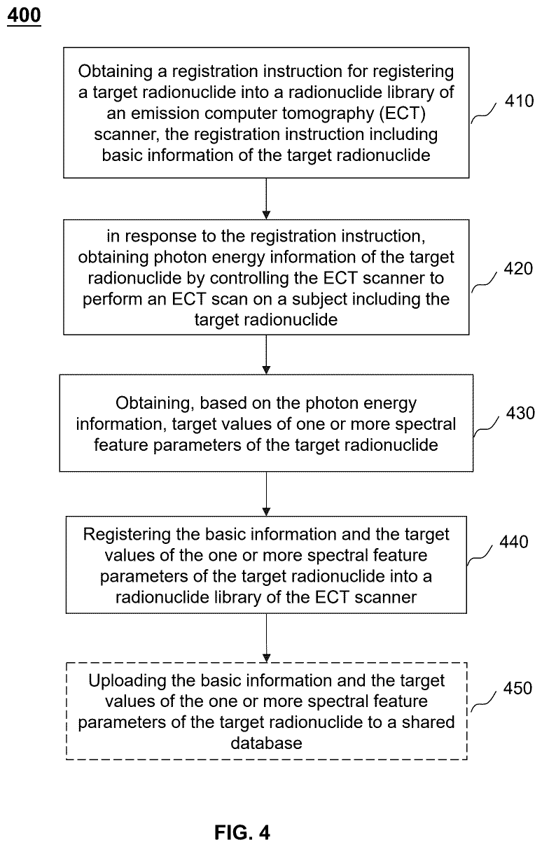

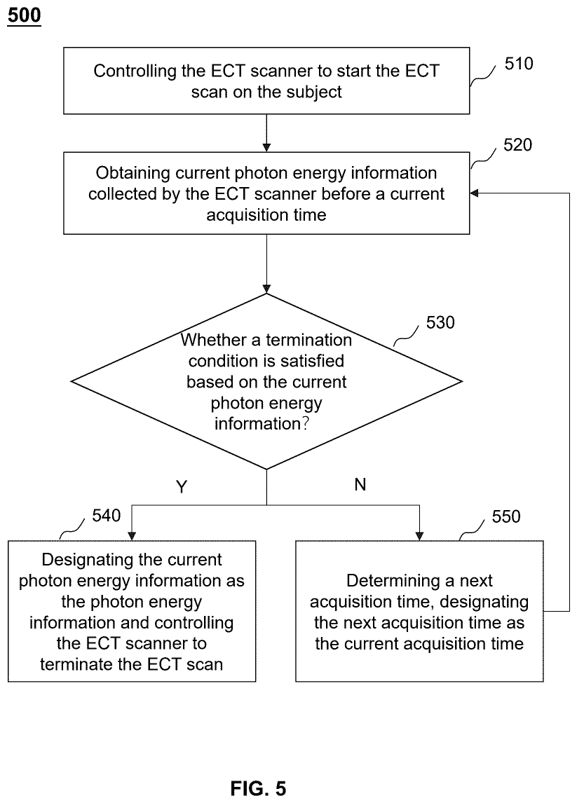

The obtaining module 310 may be configured to obtain information relating to the ECT system 100. For example, the obtaining module 310 may obtain a registration instruction for registering a target radionuclide into a radionuclide library of an ECT scanner, the registration instruction including basic information of the target radionuclide. More descriptions regarding the obtaining of the registration instruction may be found elsewhere in the present disclosure. See, e.g., operation 410 in FIG. 4, and relevant descriptions thereof. As another example, in response to the registration instruction, the obtaining module 310 may obtain photon energy information of the target radionuclide by controlling the ECT scanner to perform an ECT scan on a subject including the target radionuclide. More descriptions regarding the obtaining of the photon energy information of the target radionuclide may be found elsewhere in the present disclosure. See, e.g., operation 420 in FIG. 4, and relevant descriptions thereof.

The determination module 320 may be configured to determine target values of one or more spectral feature parameters of the target radionuclide based on the photon energy information. More descriptions regarding the determination of the target values of one or more spectral feature parameters may be found elsewhere in the present disclosure. See, e.g., operation 430 in FIG. 4, and relevant descriptions thereof.

The registering module 330 may be configured to register the basic information and the target values of the one or more spectral feature parameters of the target radionuclide into the radionuclide library of the ECT scanner. More descriptions regarding the registering of the basic information and the target values of the one or more spectral feature parameters of the target radionuclide into the radionuclide library of the ECT scanner may be found elsewhere in the present disclosure. See, e.g., operation 440 in FIG. 4, and relevant descriptions thereof.

In some embodiments, the registering module 330 may be also configured to upload the basic information and the target values of the one or more spectral feature parameters of the target radionuclide to a shared database. More descriptions regarding the upload the basic information and the target values of the one or more spectral feature parameters of the target radionuclide to the shared database may be found elsewhere in the present disclosure. See, e.g., operation 450 in FIG. 4, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, any one of the modules may be divided into two or more units. For instance, the obtaining module 310 may be divided into two units configured to acquire different data. In some embodiments, the processing device 140 may include one or more additional modules, such as a storage module (not shown) for storing data.

FIG. 4 is a flowchart illustrating an exemplary process 400 for registering radionuclide information according to some embodiments of the present disclosure.

In 410, the processing device 140 (e.g., the obtaining module 310) may obtain a registration instruction for registering a target radionuclide into a radionuclide library of an ECT scanner, the registration instruction including basic information of the target radionuclide.

The ECT scanner may be the ECT scanner 110 described in FIG. 1. The target radionuclide refers to a radionuclide whose information has not been registered in the radionuclide library.

The radionuclide library of the ECT scanner is configured to store information relating to radionuclides. The information relating to radionuclides may include basic information, values of one or more spectral feature parameters, etc., of each radionuclide. The radionuclides stored in the radionuclide library include both preset radionuclides configured by the equipment supplier of the ECT scanner and historical radionuclides subsequently registered by users during operation. The radionuclide library may be stored in a storage device (e.g., the storage device 150) using a data structure such as a list or a relational database. Before each ECT scan using the ECT scanner, a user needs to select a radionuclide to be used for this scan from the radionuclide library. Once the radionuclide is selected, the data acquisition and image reconstruction for this ECT scan will be performed based on the corresponding spectral feature parameters stored for that radionuclide in the radionuclide library.

In some embodiments, the registration instruction may include a collection instruction for causing the ECT scanner to perform an ECT scan. The registration instruction may be input by a user via various means such as voice, typing, or mouse clicking.

The basic information of the target radionuclide may include an identifier, a half-life, etc., of the target radionuclide. As used herein, an identifier of a radionuclide refers to a unique label for the radionuclide. Exemplary identifiers include a name (e.g., Indium-111) or a code (e.g., In-111, Tc-99m) of the radionuclide. A half-life of a radionuclide refers to a time required for the activity of the radionuclide to decrease to half of its initial value, typically measured in seconds. For instance, the half-life of In-111 is 2,419,200 seconds.

In 420, in response to the registration instruction, the processing device 140 (e.g., the obtaining module 310) may obtain photon energy information of the target radionuclide by controlling the ECT scanner to perform an ECT scan on a subject including the target radionuclide.

The subject may include a patient, an animal, a phantom, or a portion thereof. In some embodiments, before the ECT scan, the target radionuclide and a drug carrier for carrying the target radionuclide are introduced into the subject. For example, the subject is a patient, before the ECT scan, the target radionuclide and the drug carrier for carrying the target radionuclide are injected into the subject. Then, the ECT scanner may be controlled to start the ECT scan on the subject. In some embodiments, the subject may be positioned in advance on the couch of the ECT scanner before scanning, and adjusted to a predetermined position where a region to be scanned of the subject lies within the field of view of the ECT scanner. This positioning operation may be completed before the user inputs the registration instruction. As described in operation 410, the registration instruction may include a collection instruction, when the processing device 120 receives the registration instruction, if it detects that the subject has been positioned and is within the detection range, it may automatically control the ECT scanner to directly initiate the ECT scan, without requiring further positional adjustments or manual triggering to start the ECT scan.

The photon energy information of the target radionuclide may include information relating to photons (gamma photons) emitted from the decay of the target radionuclide during the ECT scan. The information relating to photons may include energy of each photon, a count rate of photons with the same energy, or the like. In some embodiments, the photon energy information of the target radionuclide may be presented via a graphical or numerical representation of a count rate of photons detected by the ECT scanner as a function of their energy. For example, the processing device 140 may generate an energy spectrum curve based on the photon energy information. The energy spectrum curve shows a count rate of photons detected by the ECT scanner as a function of their energy. In some embodiments, the processing device 140 may present the energy spectrum curve to the user. In some embodiments, the energy spectrum curve is presented after the ECT scan. In some embodiments, the energy spectrum curve is presented during the ECT scan.

During the ECT scan, the ECT scanner continuously acquires photons emitted from the decay of the target radionuclide within the subject and records the energy value of each detected photon. After a period of continuous acquisition, the recorded energy values are statistically analyzed to determine the photon energy information.

In some embodiments, the processing device 140 may perform the following steps (a)-(d) to obtain photon energy information of the target radionuclide. In step (a), the processing device 140 may control the ECT scanner to start the ECT scan on the subject. In step (b), the processing device 140 may obtain current photon energy information collected by the ECT scanner before a current acquisition time. In step (c), the processing device 140 may determine whether a termination condition is satisfied based on the current photon energy information. In step (d), in response to determining that the termination condition is satisfied, the processing device 140 may designate the current photon energy information as the photon energy information and controlling the ECT scanner to terminate the ECT scan; or in response to determining that the termination condition is not satisfied, the processing device 140 may determine a next acquisition time, designate the next acquisition time as the current acquisition time, and repeat steps (b) through (d). More descriptions regarding the obtaining of the photon energy information of the target radionuclide may be found elsewhere in the present disclosure (e.g., FIG. 5 and the descriptions thereof).

In 430, the processing device 140 (e.g., the determination module 320) may obtain, based on the photon energy information, target values of one or more spectral feature parameters of the target radionuclide.

The one or more spectral feature parameters of the target radionuclide may be a set of key parameters required by the ECT scanner for the ECT scan and image reconstruction. In some embodiments, the one or more spectral feature parameters may include an energy peak (also referred to as photopeak), an energy window, a scatter window, a branching ratio of the target radionuclide, or the like, or any combination thereof.

As used herein, the energy peak of the target radionuclide refers to an energy of characteristic photons emitted during the decay of the target radionuclide. For example, in PET imaging, the positrons emitted by the target radionuclide annihilate with electrons in biological tissues, producing two gamma photons traveling in opposite directions, each with an energy of 511 keV. This 511 keV energy constitutes the energy peak for the target radionuclide in PET imaging. It serves as the sole energy reference for the detector of the PET scanner to identify valid annihilation events, perform coincidence detection, and reconstruct images. As another example, in SPECT imaging, the target radionuclide directly emits gamma photons with characteristic energies during decay. The characteristic energy value of such gamma photons is defined as the energy peak for the target radionuclide in SPECT imaging. Different radionuclides exhibit distinct characteristic energy peaks. For example, the radionuclide ^{99m}Tc has an energy peak of 140 keV, while ¹²³I has an energy peak of 159 keV. This energy peak forms the core physical basis for setting energy windows and discriminating target photons. A radionuclide may have one energy peak, or more than one energy peaks.

The energy window (also referred to as a main energy window) of the target radionuclide refers to an energy range set around the energy peak of the target radionuclide. During scanning, only photons whose detected energy value falls within the energy window are recorded as valid counts. For example, in PET imaging, the energy window refers to an energy range set around 511 keV. Due to the inherent energy resolution limitations of the detector, the PET scanner cannot precisely acquire photons with energies exactly equal to 511 keV. Therefore, an energy window is set to accept photons near this energy while filtering out stray counts. Setting the lower boundary of the energy window too high results in the exclusion of a significant number of valid annihilation photons, substantially reducing system sensitivity. Conversely, setting it too low introduces excessive scattered photons, degrading image contrast and quantitative accuracy. Thus, an appropriate energy window needs to be set. As another example, in SPECT imaging, the energy window refers to an energy range set around the characteristic energy peak of the target radionuclide. This window is used to screen photons falling within a specific energy range, thereby excluding low-energy scattered photons and high-energy stray events. For example, when imaging with ^{99m}Tc, the energy window may be a range of 126 keV-154 keV. This effectively suppresses false counts and improves the signal-to-noise ratio of the resulting image. It should be noted that although an excessively narrow window can further reduce scatter, it also leads to the loss of some valid counts, thereby reducing system sensitivity. Therefore, an appropriate energy window needs to be set.

The scatter window of the target radionuclide refers to an energy window set in the vicinity of the energy peak for estimating the contribution of scattered photons to the measured data. In some embodiments, the scatter window includes a left scatter window located below the energy value corresponding to the energy peak and/or a right scatter window located above the energy value corresponding to the energy peak. By acquiring counts within the scatter window, scattered photons mixed into the main energy window can be estimated and corrected, thereby improving image quality.

For example, in PET imaging, the scatter window refers to a low-energy window specifically designed for Compton-scattered gamma photons. The 511 keV gamma photons produced by the annihilation of the target radionuclide lose part of their energy through Compton scattering as they traverse biological tissues, resulting in reduced energy and altered direction of travel. By collecting these energy-degraded scattered photons, the scatter window provides quantitative data on the scattering effect. Based on this data, a dedicated scatter-correction algorithm can be used to estimate and subtract the interference signals caused by scattering within the energy window (around 511 keV), thereby improving the clarity of the reconstructed image. As another example, in SPECT imaging, the scatter window refers to a dedicated energy range set auxiliary to the energy window for scatter correction, typically implemented using dual-window or multi-window techniques. This window is positioned outside the energy window of the target radionuclide and collects photons that originate from scattering events to estimate and correct the level of scatter interference in the energy window. For example, when imaging with ¹²³I, a scatter window (111-143 keV) can be set adjacent to the energy window (143-175 keV). By analyzing the statistical relationship between the counts in the scatter window and those in the energy window, the scatter contribution within the energy window can be estimated and subtracted. This correction technique is particularly critical in imaging scenarios where scattered photons account for as much as 30-40% of the total counts, such as in whole-body imaging or cardiac imaging, and significantly enhances lesion contrast and quantitative accuracy of reconstruction images.

The branching ratio of the target radionuclide refers to the probability that the target radionuclide emits a characteristic photon of a specific energy during a single decay event. The branching ratio is an intrinsic physical property of the target radionuclide. For example, in PET imaging, the branching ratio refers to the probability that the target radionuclide undergoes effective decay via the positron-emission decay mode. Merely by way of example, the branching ratio of the radionuclide ¹⁸F is approximately 96.7%, meaning that out of every 100 decays, an average of 96.7 occur through positron emission, while the remaining decay paths do not produce positron signals usable for imaging. This branching-ratio value is a key physical parameter for estimating the initial activity of the radiopharmaceutical required for imaging. As another example, in SPECT imaging, the branching ratio refers to the probability of the decay path in which the target radionuclide emits the characteristic gamma photons usable for imaging during its decay process. Some radionuclides for SPECT decay through multiple branches, and only one of these branches releases gamma photons whose energy and intensity meet the requirements for imaging. The occurrence probability of this specific branch constitutes the branching ratio, which is a core physical quantity that determines the ultimate detected photon flux and image signal strength. For instance, a radionuclide exhibits a gamma decay branch and a beta decay branch simultaneously. Only the gamma-decay branch releases gamma photons that can be detected by the SPECT system for imaging.

In some embodiments, a user may determine the target values of one or more spectral feature parameters of the target radionuclide based on the energy spectrum curve, and input the target values of one or more spectral feature parameters via a terminal device (e.g., the terminal device 130). The processing device 140 may obtain the target values of one or more spectral feature parameters of the target radionuclide.

In some embodiments, the processing device 140 may determine the target values of one or more spectral feature parameters of the target radionuclide based on the photon energy information using a preset algorithm.

Specifically, the processing device 140 may determine target values of one or more spectral feature parameters of the target radionuclide based on the energy spectrum curve.

In some embodiments, the processing device 140 may perform smoothing processing on the energy spectrum curve, for example, using a moving average filtering algorithm, to eliminate high-frequency noise caused by statistical fluctuations, thereby generating smooth and clear energy spectrum curve for subsequent analysis.

In some embodiments, the processing device 140 may automatically perform energy peak detection and localization on the energy spectrum curve to determine the target values of the one or more energy peaks. Specifically, the processing device 140 may search one or more significant peaks (i.e., main peaks) on the energy spectrum curve using algorithms such as a first-derivative inflection point identification algorithm, a second-derivative minimum value identification algorithm, or a Gaussian fitting algorithm. Then, for each main peak, the processing device 140 may perform a curve fitting on the main peak using a Gaussian function, determine the energy value corresponding to the center of the main peak, and designate the energy value as the target value of the energy peak. In some embodiments, the processing device 140 may determine a full width at half maximum (FWHM) of the main peak. The FWHM refers to an energy difference between the two points on the main peak where the count rate drops to half of its maximum value.

In some embodiments, the processing device 140 may determine the target value of the energy window based on the target value of the energy peak. Specifically, the processing device 140 may determine a total width of the energy window. For example, the processing device 140 may obtain a preset percentage width. The preset percentage width refers to a predetermined value, expressed as a percentage (e.g., 10%, 20%) of the target value of the energy peak. The processing device 140 may determine the product of the target value of the energy peak and the preset percentage width as the total width of the energy window. As another example, processing device 140 may determine a specific multiple (e.g., 1 multiple, 1.5 multiples, 2 multiples) of the FWHM as the total width of the energy window. Then, the processing device 140 may, using the target value of the energy peak as the center, equally divide the total width of the energy window along the energy axis to determine the lower boundary and the upper boundary of the energy window. The lower boundary is equal to the target value of the energy peak minus one-half of the total width of the energy window, and the upper boundary is equal to the target value of the energy peak plus one-half of the total width of the energy window. For example, for Tc-99m with an energy peak of 140 keV, an energy window determined with a 10% width ranges from 133 keV to 147 keV.

In some embodiments, the processing device 140 may determine the target value of the scatter window based on the target value of the energy peak. Specifically, the processing device 140 may determine a first center of the left scatter window and a second center of the right scatter window. In some embodiments, the processing device 140 may apply a preset offset relative to the target value of the energy peak. For example, the offset may be defined as a specific multiple of the FWHM (e.g., 1×FWHM, 1.5×FWHM). As another example, the offset may be defined as a percentage (e.g., 15%, 25%) of the target value of the energy peak. Accordingly, the center of the left scatter window may be positioned at an energy value equal to the target value of the energy peak minus the offset, and the center of the right scatter window may be positioned at an energy value equal to the target value of the energy peak plus the offset. The processing device 140 may determine a first width of the left scatter window and a second width of the right scatter window. For example, the first and second widths may be set to a specific multiple of the FWHM (e.g., 0.5×FWHM, 1.0×FWHM). As another example, the first and second widths may be defined as a fixed percentage (e.g., 5%, 8%) of the target value of the energy peak, or as a predetermined absolute energy value. Then, for the left scatter window, the processing device 140 may, using the first center as the reference, equally divide the first width along the energy axis to determine the lower boundary and the upper boundary of the left scatter window. For the right scatter window, the processing device 140 may, using the second center as the reference, equally divide the second width along the energy axis to determine the lower boundary and the upper boundary of the second scatter window. For example, the energy peak has a target value of 140 keV and an FWHM of 10 keV, the offset for the scatter window is preset to 1.5×FWHM (i.e., 15 keV), and the scatter window width is preset to 1.0×FWHM (i.e., 10 keV). Then, the first center is 125 keV, and the left scatter window ranges from 120 keV to 130 keV. The second center is 155 keV, and the right scatter window ranges from 150 keV to 160 keV.

In some embodiments, the branching ratio may be directly provided by the user and included in the registration instruction. In some embodiments, if the target radionuclide has multiple energy peaks (e.g., In-111), the processing device 140 can calculate the corresponding count area (total counts) corresponding to each energy peak. Subsequently, by comparing these count areas in combination with the pre-stored relative detection efficiency of the ECT scanner at different energies, the processing device 140 derives the branching ratio corresponding to each energy peak.

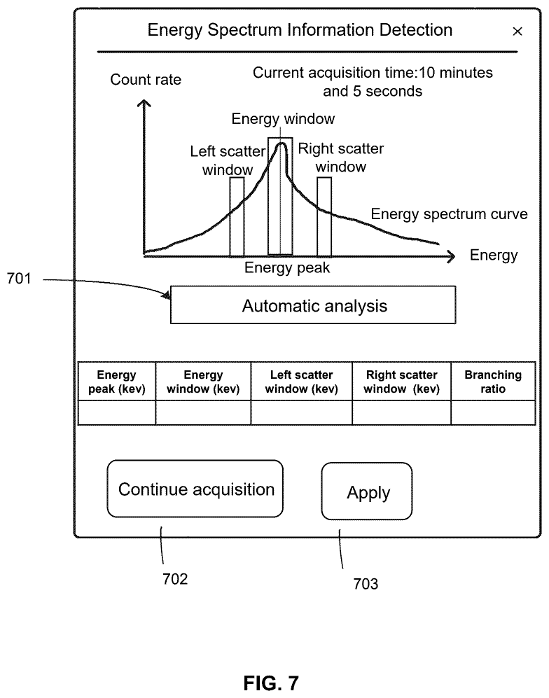

FIG. 7 is a schematic diagram illustrating an exemplary interface 700 for energy spectrum information detection according to some embodiments of the present disclosure.

As shown in FIG. 7, after obtaining the photon energy information, an energy spectrum curve can be generated, with an energy as the horizontal axis and a count rate as the vertical axis. The energy peak, the energy window, the left scatter window, and the right scatter window may be displayed on the energy spectrum curve after determining the target values for the energy peak, the energy window, the left scatter window, the right scatter window, and the branching ratio. In some embodiments, the energy spectrum curve is presented to a user. The user may click the automatic analysis button 701 as shown in FIG. 7. In response to this operation, the target values for the energy peak, the energy window, the branching ratio, the left scatter window, and the right scatter window can be displayed. As illustrated in FIG. 7, the current acquisition time is 10 minutes and 5 seconds, the target values of the energy peak, the branching ratio, the energy window, the left scatter window, and the right scatter window are shown in a table.

In some embodiments, the processing device 140 may determine a plurality of recommendation sets based on the photon energy information. Each recommendation set includes recommended values of the one or more spectral feature parameters. In some embodiments, the plurality of recommendation sets may include a basic recommendation set, a high-resolution recommendation set, a high-sensitivity recommendation set, a custom recommendation set, or the like, or any combination thereof. The basic recommendation set refers to standard values of the one or more spectral feature parameters automatically calculated based on the photon energy information. Based on the basic recommendation set, the high-resolution recommendation set is obtained by reducing the width of the energy window in the basic recommendation set, thereby improving image resolution and reducing scatter impact. If the target radionuclide has multiple energy peaks (e.g., In-111), for each energy peak, an energy window is separately configured and enabled based on the basic recommendation set, so that photon counts from all enabled energy windows are collected for image reconstruction to enhance detection sensitivity and reduce image noise. The custom recommendation set refers to a set of values for the one or more spectral feature parameters defined by the user through manual input or adjustment.

For each recommendation set, the processing device 140 may reconstruct an ECT image of the subject based on ECT data collected by the ECT scanner in the ECT scan and the recommended values in the recommendation set. Specifically, the processing device 140 may reconstruct the ECT image of the subject based on the ECT data and the recommended values using a reconstruction algorithm. Exemplary reconstruction algorithms may include an analytic reconstruction algorithm (e.g., a filtered back projection (FBP), a Fourier reconstruction algorithm), an iterative reconstruction algorithm (e.g., a maximum likelihood expectation maximization (MLEM), an ordered subsets expectation maximization (OSEM)), a hybrid and deep learning-based algorithm (e.g., a deep learning reconstruction algorithm), or the like.

Further, the processing device 140 may present the ECT image and the recommended values corresponding to each recommendation set to the user. For example, the processing device 140 may display the ECT image corresponding to each recommendation set side-by-side or in a paginated layout within the same display interface. Each ECT image is associated with and displays its corresponding recommended values. Based on a visual assessment of the ECT images, the user selects one recommendation set as a target recommendation set from the plurality of recommendation sets via an interactive interface (e.g., through clicking or touch-selection operations). The visual assessment may include an assessment for clarity in a lesion region, an assessment for background noise level, an assessment for overall contrast, or the like, or any combination thereof.

In some embodiments, the processing device 140 may determine one or more quality factors of the ECT image corresponding to each recommendation set. The quality factors may include a signal-to-noise ratio, a contrast, a uniformity, or the like, or any combination thereof. Further, the processing device 140 may present the ECT image, the recommended values, and the one or more quality factors corresponding to each recommendation set to the user. The user may select one recommendation set as the target recommendation set from the plurality of recommendation sets based on the visual assessment and the one or more quality factors.

Then, in response to a selection instruction for selecting the target recommendation set from the recommendation sets, the processing device 140 may designate the recommended values in the target recommendation set as the target values of the one or more spectral feature parameters. In some embodiments, the user may adjust the recommended values in the one recommendation set to generate a candidate recommendation set. The processing device 140 may reconstruct a candidate ECT image of the subject based on ECT data and the recommended values in the candidate recommendation set. Then, the processing device 140 may present the candidate ECT image and the recommended values corresponding to the candidate recommendation set to the user. The user is prompted to confirm whether to accept the candidate recommendation set as the target recommendation set.

According to some embodiments of the present disclosure, the target values of the one or more spectral feature parameters may be determined based on the ECT images corresponding to the recommendation sets and the recommended values of the one or more spectral feature parameters. In this way, the target values of the one or more spectral feature parameters can ensure the reconstruction of ECT images with optimal clinical diagnostic value, thereby resolving the disconnect between theoretically calculated values of the one or more spectral feature parameters and actual image quality inherent in traditional methods. Moreover, by presenting the ECT images corresponding to the recommendation sets in parallel, along with their quality factors, the process of selecting the target recommendation set is transformed from one dependent on subjective experience to a visual, comparable, and quantifiable procedure. This significantly enhances the repeatability, consistency, and scientific basis of spectral feature parameter configuration while reducing fluctuations in imaging quality caused by individual experiential differences. In addition, while providing intelligent recommendations, some embodiments of the present disclosure retain the capability for users to manually adjust parameters and preview imaging effects in real time. This achieves a “tailor-made” approach to spectral feature parameter configuration.

In 440, the processing device 140 (e.g., the registering module 330) may register the basic information and the target values of the one or more spectral feature parameters of the target radionuclide into the radionuclide library of the ECT scanner.

For example, the processing device 140 adds an entry corresponding to the target radionuclide to the radionuclide library, and records the basic information and the target values of the one or more spectral feature parameters of the target radionuclide in a preset format into the radionuclide library. Upon completion of the registration, the target radionuclide is included in the radionuclide library for user selection. In subsequent ECT scans, the user only needs to select the target radionuclide, and its corresponding target values of the spectral feature parameters will be automatically invoked for data acquisition and image reconstruction.

In some embodiments, the processing device 140 may present the energy spectrum curve and the target values of the one or more spectral feature parameters to the user. In response to a confirmation instruction of the target values, the processing device 140 may register the basic information and the target values of the one or more spectral feature parameters of the target radionuclide into the radionuclide library of the ECT scanner.

Merly by way of example, as shown in FIG. 7, after reviewing the target values for the energy peak, the energy window, the branching ratio, the left scatter window, and the right scatter window, if the user is concerned about potential inaccuracies and wishes to continue acquisition, they may click the continue acquisition button 702 in FIG. 7. This action may control the ECT scanner to resume scanning. Alternatively, if the user confirms that the results are correct, they may click the apply button 703 to confirm the target values. The processing device 140 may register the basic information and the target values of the one or more spectral feature parameters of the target radionuclide into the radionuclide library of the ECT scanner.

In some embodiments, the processing device 140 may present a drug carrier list including candidate drug carriers. A drug carrier can bind with a radionuclide to form a radiopharmaceutical. During an ECT scan, the radiopharmaceutical is introduced into the scanned subject. The drug carrier is responsible for transporting the radionuclide to specific target parts within the body (e.g., a tumor, an inflammatory part, a specific organ, or physiological pathways). Exemplary drug carriers include fluorine-18 (18F), fluorodeoxyglucose (FDG), peptides, antibodies, etc. For example, Capromab Pendetide can serve as a drug carrier for In-111 to form the radiopharmaceutical In-111 Capromab Pendetide, used for prostate cancer imaging. The processing device 140 may obtain a selection instruction for selecting one or more target drug carriers from the drug carrier list. Then, the processing device 140 may register associated information between the target radionuclide and the one or more target drug carriers into the radionuclide library.

Before an ECT scan, an associated drug carrier list for the target radionuclide can be presented to the user to select a target drug carrier for use in the ECT scan from the one or more target drug carriers.

After the target radionuclide is registered in the radionuclide library, it may be selected for use in subsequent ECT scans of other subjects. For example, the processing device 140 may obtain a selection instruction for selecting the target radionuclide for a second subject from the radionuclide library. The processing device 140 may obtain third photon energy information collected by the ECT scanner in a second ECT scan on the second subject. In some embodiments, the second ECT scan may be a pre-scan with the same acquisition duration as the ECT scan described in connection with operation 420. Alternatively, the second ECT scan is a formal ECT scan during which the third photon energy information is collected at its start using the same acquisition duration as the ECT scan described in connection with operation 420.

The processing device 140 may determine whether the selection instruction is accurate based on the target values of the one or more spectral feature parameters and the third photon energy information. Specifically, the processing device 140 may determine second values of the one or more spectral feature parameters based on the third photon energy information. In some embodiments, the processing device 140 may determine the second values of the one or more spectral feature parameters based on the third photon energy information in a similar manner as how to determine the target values of the one or more spectral feature parameters described in operation 430, and the descriptions thereof are not repeated here. Further, for each spectral feature parameter, the processing device 140 may determine a difference between the second value and the target value of the spectral feature parameter. In response to determining that there is no spectral feature parameter whose difference is greater than the difference threshold, the processing device 140 may determine that the selection instruction is accurate. In response to determining that there is one or more spectral feature parameters whose differences are greater than a difference threshold, the processing device 140 may determine that the selection instruction is likely to be inaccurate and generate a prompt. The prompt may be issued to the user by a terminal device and used to remind the user to check whether the selection instruction is accurate and confirm whether the correct radionuclide has been introduced for the second subject. In this way, the risk of the user selecting or introducing the wrong radionuclide can be avoided.

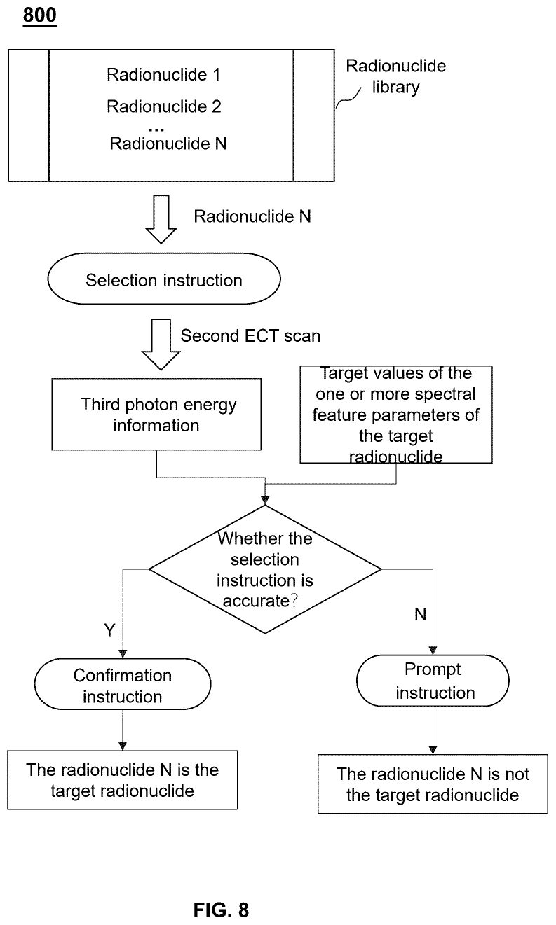

For example, FIG. 8 is a schematic diagram illustrating an exemplary process 800 for confirming values of spectral feature parameters for an ECT scan according to some embodiments of the present disclosure. As shown in FIG. 8, the radionuclide library of the ECT scanner stores information relating to radionuclides 1, 2, . . . , N. The processing device 140 obtains a selection instruction for selecting the radionuclide N for a second subject from the radionuclide library. A second ECT scan is performed on the second subject based on values of spectral feature parameters of the radionuclide N stored in the radionuclide library. The processing device 140 obtains third photon energy information collected by the ECT scanner in the second ECT scan on the second subject. The processing device 140 determines whether the selection instruction is accurate based on the target values of the one or more spectral feature parameters and the third photon energy information. In response to determining that the selection instruction is accurate, the processing device 140 generates a confirmation instruction. The confirmation instruction indicates that the radionuclide N is the target radionuclide. In response to determining that the selection instruction is not accurate, the processing device 140 generates a prompt instruction for controlling a terminal device to issue a prompt.

In 450, the processing device 140 (e.g., the registering module 330) may upload the basic information and the target values of the one or more spectral feature parameters of the target radionuclide to a shared database.

The shared database is configured to store radionuclide information accessible to authorized users (e.g., within the same hospital). For example, when the target radionuclide needs to be registered on other ECT scanners in the hospital, the processing device 140 may retrieve the shared database to obtain the basic information and the target values of the one or more spectral feature parameters of the target radionuclide, and directly register them onto those ECT scanners. This eliminates the need for the repetitive configuration work across different ECT scanners within the same hospital.

In some embodiments, the ECT scan includes a first scan period and a second scan period, and the first scan period is earlier than the second scan period. The processing device 140 may obtain the target values of the one or more spectral feature parameters of the target radionuclide based on scan data collected during the first scan period. Specifically, the processing device 140 may perform operation 430 to obtain the target values of the one or more spectral feature parameters of the target radionuclide. Then, the processing device 140 may reconstruct an ECT image of the subject based on the target values of one or more spectral feature parameters of the target radionuclide and scan data collected the second scan period. In some embodiments, the processing device 140 reconstructs the ECT image of the subject according to the target values of one or more spectral feature parameters of the target radionuclide and the scan data collected during the first scan period and the second scan period. In some embodiments, the processing device 140 reconstructs the ECT image of the subject according to the target values of one or more spectral feature parameters of the target radionuclide and the scan data collected the second scan period. Specifically, the processing device 140 may reconstruct the ECT image of the subject using a reconstruction algorithm (e.g., the reconstruction algorithms described in operation 430). In some embodiments, the processing device 140 may register the target values of the one or more spectral feature parameters of the target radionuclide obtained based on the scan data collected during the first scan period into the radionuclide library of the ECT scanner.

Specifically, in some embodiments, during the first scan period, the processing device 140 may perform process 400 to register the basic information and the target values of the one or more spectral feature parameters of the target radionuclide into the radionuclide library of the ECT scanner. That is, the photon energy information of the target radionuclide described in operation 420 is obtained based on the scan data collected in the first scan period. In this case, after the ECT scan of the first scan period is completed, it does not terminate but continues with the ECT scan of the second scan period. That is, in operation 540, the processing device 140 may designate the current photon energy information as the photon energy information and control the ECT scanner to continue the ECT scan of the second scan period. Then, the processing device 140 may reconstruct the ECT image of the subject based on the target values of one or more spectral feature parameters of the target radionuclide and the scan data collected the second scan period.

As described elsewhere in the present disclosure, conventional approaches for registering radionuclide information suffer from low efficiency and a high risk of human error, which may consequently lead to degraded imaging quality and reduced diagnostic accuracy. Compared with the conventional approaches, the process 400 can rapidly determine accurate values of the spectral feature parameters of the target radionuclide. Moreover, the process 400 may be automatically implemented with reduced or minimal or without user intervention, which is more efficient (by, e.g., reducing the workload of a user and the time needed for registering radionuclide information).

FIG. 5 is a flowchart illustrating an exemplary process 500 for obtaining photon energy information of a target radionuclide according to some embodiments of the present disclosure.

In 510, the processing device 140 may control the ECT scanner to start the ECT scan on the subject.

Before the ECT scan, the target radionuclide and a drug carrier for carrying the target radionuclide are introduced into the subject. Then, the ECT scanner may be controlled to start the ECT scan on the subject, and the processing device 140 may initiate a timer.

In 520, the processing device 140 may obtain current photon energy information collected by the ECT scanner before a current acquisition time.

In some embodiments, the current acquisition time may be any time after the ECT scan starts, such as 1 minute, 2 minutes, 8 minutes, etc.

In some embodiments, the current acquisition time is determined based on a preset acquisition time sequence including a plurality of acquisition times arranged in ascending order. As used herein, an acquisition time (e.g., the current acquisition time, the next acquisition time described in operation 550) refers to a specific point in time measured from the start of the ECT scan. The current acquisition time may be any one among the plurality of acquisition times. In some embodiments, a time interval between two consecutive acquisition times is equal. For example, the time interval between two consecutive acquisition times may be 30 seconds, 1 minute, 2 minutes, etc.

The current photon energy information includes photon energy information collected by the ECT scanner from the start of the ECT scan up to the current acquisition time. For example, if the current acquisition time is 10 minutes, the current photon energy information includes photon energy information collected over these 10 minutes. As another example, if the current acquisition time is 15 minutes, the current photon energy information includes photon energy information collected over these 15 minutes.

In some embodiments, the preset acquisition time sequence may be set manually by a user (e.g., a radiologist) according to an experience value or a default setting of the ECT system 100. For example, the preset acquisition time sequence includes acquisition times 5 minutes, 6 minutes, 7 minutes, 8 minutes, 9 minutes, 10 minutes, 11 minutes, 12 minutes, and 13 minutes. As another example, the preset acquisition time sequence includes 10 minutes, 11 minutes, 12 minutes, 13 minutes, 14 minutes, and 15 minutes.

In some embodiments, the basic information of the target radionuclide includes a half-life period or the branching ratio of the target radionuclide, and the registration instruction further includes an initial activity of the target radionuclide. As used herein, the initial activity refers to an activity of the target radionuclide when the target radionuclide is introduced into the subject. The preset acquisition time sequence may be determined based on the initial activity and the half-life period or the branching ratio of the target radionuclide.

In some embodiments, the processing device 140 may determine the first acquisition time in the preset acquisition time sequence and the time interval between two consecutive acquisition times in the preset acquisition time sequence based on the initial activity and the half-life period or the branching ratio of the target radionuclide. Then, the processing device 140 may determine the preset acquisition time sequence based on the first acquisition time, the time interval between two consecutive acquisition times, and a maximum total duration. The maximum total duration refers to the maximum allowable duration of the ECT scan. For example, if the first acquisition time is 5 minutes, the time interval is 2 minutes, and the maximum total duration is 13 minutes, the processing device 140 may determine that the preset acquisition time sequence includes 5 minutes, 7 minutes, 9 minutes, 11 minutes, 13 minutes, etc.

In some embodiments, the processing device 140 may determine the first acquisition time and the time interval between two consecutive acquisition times based on the initial activity and the half-life period of the target radionuclide. The greater the initial activity, the shorter the first acquisition time. The shorter the half-life period, the shorter the first acquisition time. In some embodiments, the processing device 140 determines the first acquisition time and the time interval based on a pre-stored mapping relationship. The mapping relationship defines an association rule between the half-life period, the initial activity, the first acquisition time, and the time interval.

For example, the mapping relationship includes the following association rules: if the target radionuclide has a high initial activity and/or a short half-life, the first acquisition time is a first value and the time interval is a second value; if the target radionuclide has a medium initial activity and a medium half-life, the first acquisition time is a third value and the time interval is a fourth value; if the target radionuclide has a low initial activity and a long half-life, the first acquisition time is a fifth value and the time interval is a sixth value. Here, the first value is smaller than the third value, the third value is smaller than the fifth value, the second value is smaller than the fourth value, and the fourth value is smaller than the sixth value. The first value, the second value, the third value, the fourth value, the fifth value, and the sixth value may be preset based on clinical experience or obtained from statistical analysis of historical acquisition data. The initial activity and the half-life may be classified into different levels (e.g., high, medium, or low) according to preset thresholds. The processing device 140 determines the first acquisition time and the time interval by querying the pre-stored mapping relationship.

In some embodiments, the processing device 140 may determine the first acquisition time based on the initial activity and the branching ratio of the target radionuclide. Specifically, the processing device 140 may obtain a target count threshold and a geometric efficiency of the ECT scanner, and determine the first acquisition time based on the initial activity, the branching ratio, the target count threshold, and the geometric efficiency.

The target count threshold refers to a predefined count rate that the ECT scan needs to achieve to obtain images meeting diagnostic quality requirements. The geometric efficiency of the ECT scanner is an average value of the geometric efficiencies of a plurality of points inside the subject. The geometric efficiency of a point refers to the probability that a gamma photon emitted from the point inside the subject can be detected by the detector. The primary factor reducing geometric efficiency is attenuation—the absorption or scattering of the photon by the subject itself. The thicker or denser the material along the photon's attenuation path, the greater the attenuation and the lower the geometric efficiency. In some embodiments, the processing device may determine the geometric efficiency of a point according to an empirical model. For example, multiple standard radioactive sources with known activity, shape, and size are used in scanning experiments to collect extensive detection data. Based on the detection data, a relationship between the geometric efficiency, the spatial location of the point relative to the detector, and the attenuation path between the point and the detector is established to form the empirical model. The empirical model may be pre-stored in the ECT scanner. The processing device 140 may query the empirical model and input the position information and the attenuation path information of each point into the empirical model to determine the geometric efficiency of the point.

For example, the processing device 140 may determine the first acquisition time according to the Equation (1) as below:

$${T}_{1} = \frac{{N}_{t a r g e t}}{ε {A}_{0} {B}_{r}} , \qquad ( 1 )$$

where, T₁ denotes first acquisition time, N_{target} denotes the target count threshold, ε denotes the geometric efficiency of the ECT scanner, A₀ denotes the initial activity, and Bᵣ denotes the branching ratio.

In 530, the processing device 140 may determine whether a termination condition is satisfied based on the current photon energy information.

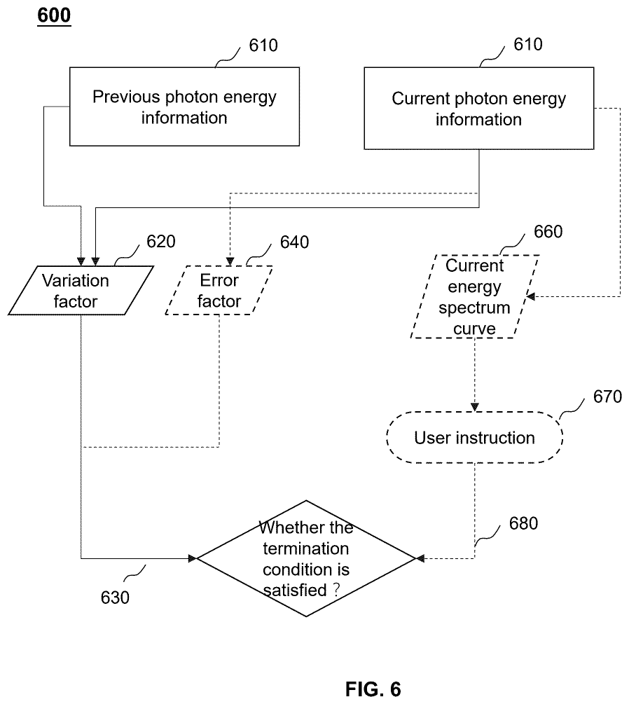

In some embodiments, the processing device 140 may obtain previous photon energy information collected by the ECT scanner before a previous acquisition time of the current acquisition time. Further, the processing device 140 may determine whether the termination condition is satisfied based on the previous photon energy information and the current photon energy information. Specifically, the processing device 140 may determine a variation factor between the previous photon energy information and the current photon energy information. Further, the processing device 140 may determine whether the termination condition is satisfied based on the variation factor. More descriptions regarding the determination of the variation factor and whether the termination condition is satisfied based on the variation factor may be found elsewhere in the present disclosure (e.g., FIG. 6 and the descriptions thereof).

In some embodiments, the processing device 140 may determine an error factor relating to one or more key energy peaks based on the current photon energy information. Further, the processing device 140 may determine whether the termination condition is satisfied based on the error factor and the variation factor. More descriptions regarding the determination of the error factor and whether the termination condition is satisfied based on the error factor and the variation factor may be found elsewhere in the present disclosure (e.g., FIG. 6 and the descriptions thereof).

In some embodiments, the processing device 140 may present an energy spectrum curve (also referred to as a current energy spectrum curve) corresponding to the current photon energy information to a user. In some embodiments, the processing device 140 may present the current energy spectrum curve and a previous energy spectrum curve corresponding to the previous photon energy information to the user. The user may determine whether the current energy spectrum curve satisfies requirements according to experience. In response to determining that the current energy spectrum curve satisfies requirements, the user transmits a confirmation instruction of the current energy spectrum curve. In response to determining that the current energy spectrum curve does not satisfy requirements, the user transmits a re-collection instruction. In response to the confirmation instruction of the energy spectrum curve, the processing device 140 may determine that the termination condition is satisfied; or in response to the re-collection instruction, the processing device 140 may determine that the termination condition is not satisfied.

In response to determining that the termination condition is satisfied, operation 540 is performed. In response to determining that the termination condition is not satisfied, operation 550 is performed. In some embodiments, if the current acquisition time is the last acquisition time in the preset acquisition time sequence and the termination condition is not satisfied, the processing device 140 generates a prompt to a user. The prompt may be issued to the user by a terminal device and used to remind the user to adjust the maximum total duration of the preset acquisition time sequence.

In 540, the processing device 140 may designate the current photon energy information as the photon energy information and control the ECT scanner to terminate the ECT scan.

In 550, the processing device 140 may determine a next acquisition time, designate the next acquisition time as the current acquisition time, and repeat operations 520-550.

In some embodiments, the next acquisition time is determined based on the preset acquisition time sequence. For example, the next acquisition time is an acquisition time next to the current acquisition time in the preset acquisition time sequence. Merely by way of example, if the preset acquisition time sequence includes acquisition times of 5 minutes, 6 minutes, 7 minutes, 8 minutes, 9 minutes, 10 minutes, 11 minutes, 12 minutes, and 13 minutes, and the current acquisition time is 8 minutes, the next acquisition time is 9 minutes.

In some embodiments, the next acquisition time is determined based on the error factor and the variation factor. Specifically, the processing device 140 determines a preset next acquisition time based on the preset acquisition time sequence. For example, the preset next acquisition time is an acquisition time adjacent to the current acquisition time in the preset acquisition time sequence. Further, the processing device 140 determines the next acquisition time by adjusting the preset next acquisition time based on the error factor and the variation factor.

In some embodiments, the processing device 140 determines whether the error factor is smaller than an error threshold (also referred to as a first error threshold) and the variation factor is smaller than a variation threshold (also referred to as a first variation threshold). The error threshold and the variation threshold may be set manually by a user (e.g., a radiologist) according to an experience value or be a default setting of the ECT system 100. If both the error factor and the variation factor are below their respective thresholds, the processing device 140 may determine the next acquisition time by subtracting a reduction value from the preset next acquisition time. The reduction value scales with the extent to which each factor is under its threshold. If both the error factor and the variation factor are greater than their respective thresholds, the processing device 140 may determine the next acquisition time by adding an addition value from the preset next acquisition time. The addition value scales with the extent to which each factor is under its threshold.

In some embodiments, the processing device 140 determines the next acquisition time based on the error factor, the variation factor, and a half-life decay model of the target radionuclide. Specifically, the processing device 140 retrieves a pre-stored half-life decay model of the target radionuclide. The processing device 140 simulates the trend of count rate decreasing over time after the current acquisition time due to the decay of the target radionuclide according to the half-life decay model. The processing device 140 determines a predicted additional acquisition duration based on the trend of the count rate decreasing over time. The additional acquisition duration is the extra time required for continuous acquisition from the current acquisition time onward such that the error factor and variation factor corresponding to the cumulatively acquired data both reach their respective preset thresholds (i.e., the error threshold and the variation threshold). Finally, the processing device 140 determines the next acquisition time as the sum of the current acquisition time and the additional acquisition duration.

According to some embodiments, the next acquisition time is determined based on the error factor and the variation factor. This can adaptively optimize the next acquisition time based on real-time feedback on data quality and stability indicators, thereby seeking the optimal acquisition efficiency while ensuring data reliability.

According to the process 500 in FIG. 5, the acquired photon energy information can satisfy the quality requirements in terms of statistical significance and stability, thereby providing an accurate and reliable data foundation for the subsequent automatic calculation of the target values of spectral feature parameters. Simultaneously, process 500 can effectively avoid two inefficient extremes inherent in traditional fixed-duration acquisition modes: first, an acquisition duration that is too short, which leads to excessive statistical fluctuations and substandard data quality, requiring rescanning and thereby reducing overall workflow efficiency; second, an acquisition duration that is too long, resulting in the unnecessary occupation of equipment time and medical staff's operational time, which wastes valuable medical resources. Through process 500, the overall time efficiency and resource utilization efficiency of the ECT scanning workflow are significantly enhanced while ensuring data usability.

It should be noted that the processes 400 and 500 and the descriptions thereof are provided for the purposes of illustration, and not intended to limit the scope of the present disclosure. For persons having ordinary skills in the art, various modifications and changes in the forms and details of the application of the above method and system may occur without departing from the principles of the present disclosure. However, those variations and modifications also fall within the scope of the present disclosure. For example, the operations of the illustrated processes 400 and 500 are intended to be illustrative. In some embodiments, the processes 400 and 500 may be accomplished with one or more additional operations not described, and/or without one or more of the operations discussed. For example, operation 450 may be omitted. Additionally, the order in which the operations of the processes 400 and 500 and regarding descriptions are not intended to be limiting.

FIG. 6 is a schematic diagram illustrating an exemplary process 600 for determining whether a termination condition is satisfied according to some embodiments of the present disclosure.

As illustrated in FIG. 6, in some embodiments, the processing device 140 may determine whether the termination condition is satisfied by performing operations 610-630.

Specifically, in 610, the processing device 140 may obtain the current photon energy information and previous photon energy information. The current photon energy information is collected by the ECT scanner before the current acquisition time, and the previous photon energy information is collected by the ECT scanner before a previous acquisition time of the current acquisition time. For example, the processing device 140 obtains the current photon energy information and the previous photon energy information from the ECT scanner or the storage 150.

In 620, the processing device 140 may determine a variation factor between the previous photon energy information and the current photon energy information. The variation factor may be used to measure the change in the current photon energy information relative to the previous photon energy information. The variation factor may include a shift of one or more key energy peaks, a change in peak height of the one or more key energy peaks, a correlation coefficient of energy spectrum curves, or the like, or any combination.

The one or more key energy peaks refer to the most central or user-interested energy peaks in the current photon energy information, which can be specified by the user or determined according to preset rules. For example, one or more energy peaks with corresponding count rates greater than a count rate threshold may be selected as the one or more key energy peaks. The shift of a key energy peak refers to a difference between the value of the key energy peak in the current photon energy information and its value in the previous photon energy information. In some embodiments, the shift of the one or more key energy peaks may be an average of the shifts of the one or more key energy peaks. In some embodiments, the shift of the one or more key energy peaks may be a maximum of the shifts of the one or more key energy peaks. The change in peak height of a key energy peak refers to a change between the count rate corresponding to the key energy peak in the current photon energy information and the count rate corresponding to the same key energy peak in the previous photon energy information. In some embodiments, the change in peak height of the one or more key energy peaks may be an average of the changes in peak height of the one or more key energy peaks. In some embodiments, the change in peak height of the one or more key energy peaks may be a maximum of the changes in peak height of the one or more key energy peaks. The correlation coefficient of the energy spectrum curves may be used to measure the similarity in overall shape between the current energy spectrum curve and the previous energy spectrum curve.

In some embodiments, to more robustly assess the variation trend of photon energy information, the calculation of the variation factor may be extended to a longer time series. For example, the processing device 140 may further obtain a second-to-last photon energy information collected by the ECT scanner before an acquisition time immediately preceding the previous acquisition time in the preset acquisition time sequence. The processing device 140 may determine a first variation factor between the previous photon energy information and the current photon energy information, and a second variation factor between the previous photon energy information and the second-to-last photon energy information. Further, the processing device 140 may determine the variation factor according to the first variation factor and the second variation factor. For example, the processing device 140 may determine an average of the first variation factor and the second variation factor as the variation factor. As another example, the processing device 140 may determine the variation factor by determining a weighted sum of the first variation factor and the second variation factor. The weight of the first variation factor is higher than the weight of the second variation factor.

In some embodiments, the processing device 140 may determine a variation factor between the second-to-last photon energy information and the current photon energy information as the variation factor. For example, the preset acquisition time sequence includes 10 minutes, 11 minutes, 12 minutes, 13 minutes, 14 minutes, and 15 minutes. If the current acquisition time is 11 minutes, the processing device 140 may determine a variation factor between the photon energy information corresponding to the 10 minutes and the current photon energy information corresponding to the 11 minutes as the variation factor. If the current acquisition time is 12 minutes, the processing device 140 may determine a variation factor between the photon energy information corresponding to the 10 minutes and the current photon energy information corresponding to the 12 minutes as the variation factor. If the current acquisition time is 13 minutes, the processing device 140 may determine a variation factor between the photon energy information corresponding to the 11 minutes and the current photon energy information corresponding to the 13 minutes as the variation factor. If the current acquisition time is 14 minutes, the processing device 140 may determine a variation factor between the photon energy information corresponding to the 12 minutes and the current photon energy information corresponding to the 14 minutes as the variation factor. If the current acquisition time is 15 minutes, the processing device 140 may determine a variation factor between the photon energy information corresponding to the 13 minutes and the current photon energy information corresponding to the 15 minutes as the variation factor.

In 630, the processing device 140 may determine whether the termination condition is satisfied based on the variation factor. Specifically, the processing device 140 may determine whether the variation factor is smaller than a second variation threshold. The second variation threshold may be set manually by a user (e.g., a radiologist) according to an experience value or be a default setting of the ECT system 100. For example, for the shift of one or more key energy peaks, the second variation threshold may be 0.5 keV, 0.6 keV, etc. For the change in the peak height of the one or more key energy peaks, the second variation threshold may be 2%, 1%, etc. For the correlation coefficient of energy spectrum curves, the second variation threshold may be 0.999, 0.991, etc. In response to determining that the variation factor is smaller than the second variation threshold, the processing device 140 may determine that the termination condition is satisfied. In response to determining that the variation factor is not smaller than the second variation threshold, the processing device 140 may determine that the termination condition is not satisfied.

As illustrated in FIG. 6, in some embodiments, the processing device 140 may further perform 640 to determine an error factor relating to one or more key energy peaks based on the current photon energy information. The error factor may be used to measure the accuracy of relevant measurement values of the one or more key energy peaks in the current photon energy information. A smaller error factor indicates higher measurement precision and less influence from statistical fluctuations. The error factor may include a relative standard error (RSE) of a net count rate corresponding to the one or more key energy peaks. The net count rate of a key energy peak refers to a count rate after subtracting background count rates (e.g., a Compton scatter count rate, an electronic noise count rate). For example, the processing device 140 may determine the RSE according to equation (2) as below:

$$R S E = \frac{1}{\sqrt{{N}_{n e t}}} , \qquad ( 2 )$$

where, the Nₙₑₜ denotes the net count rate (e.g., an average net count rate) corresponding to the one or more key energy peaks.

If the error factor is determined, the processing device 140 may determine whether the termination condition is satisfied based on the error factor and the variation factor in operation 630. Specifically, the processing device 140 may determine whether the error factor is smaller than a second error threshold. The second error threshold may be set manually by a user (e.g., a radiologist) according to an experience value or be a default setting of the ECT system 100. For example, for the RSE, the second error threshold may be 2%, 3%, 5%, etc. In response to determining that the error factor is smaller than the second error threshold and the variation factor is smaller than the second variation threshold, the processing device 140 may determine that the termination condition is satisfied. In response to determining that the error factor is not smaller than the second error threshold or the variation factor is not smaller than the second variation threshold, the processing device 140 may determine that the termination condition is not satisfied.

As illustrated in FIG. 6, in some embodiments, the processing device 140 may determine whether the termination condition is satisfied by performing operations 610 and 660-680.

Specifically, in 660, the processing device 140 may present the current energy spectrum curve corresponding to the current photon energy information to the user. In some embodiments, the processing device 140 may present the current energy spectrum curve and the previous energy spectrum curve to the user.

In 670, the processing device 140 may receive a user instruction. For example, if the user determines that the current energy spectrum curve satisfies requirements, the user transmit a confirmation instruction; if the user determines the current energy spectrum curve does not satisfy requirements, the user transmit a re-collection instruction.

In 680, the processing device 140 determines whether the termination condition is satisfied based on the user instruction. In response to the confirmation instruction, the processing device 140 may determine that the termination condition is satisfied; or in response to the re-collection instruction, the processing device 140 may determine that the termination condition is not satisfied.

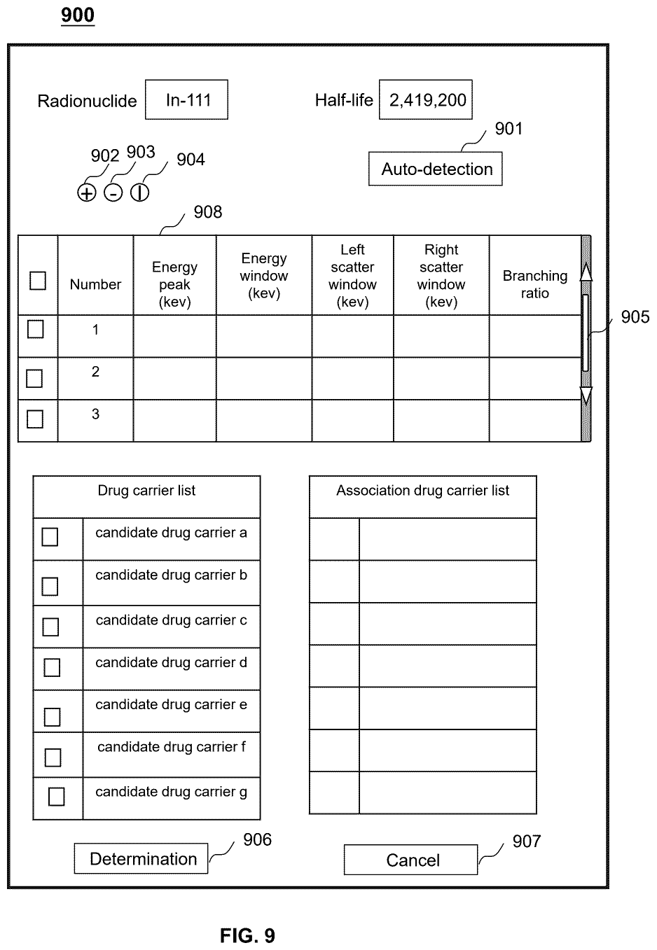

FIG. 9 is a schematic diagram illustrating an exemplary interface 900 for registering radionuclide information according to some embodiments of the present disclosure.

A user enters basic information (e.g., the name, the half-life) of the target radionuclide via the interface 900. For example, as shown in FIG. 9, the name of the target radionuclide is In-111, and its half-life is 2,419,200 seconds. The user may then click an “auto-detection” button 901 to input a registration instruction. In some embodiments, in response to the registration instruction, the processing device 140 queries whether the target radionuclide exists in a shared database. In response to confirming the existence of the target radionuclide, the processing device 140 retrieves and registers the basic information and values of the target radionuclide from the shared database into the radionuclide library of the ECT scanner, bypassing subsequent steps. In response to confirming the absence of the target radionuclide, the following steps are executed to determine the radionuclide information for the target radionuclide.

In response to the registration instruction, the processing device 140 may obtain photon energy information of the target radionuclide by controlling the ECT scanner to perform an ECT scan on a subject including the target radionuclide.

Further, the processing device 140 may determine target values of one or more spectral feature parameters of the target radionuclide based on the photon energy information. The processing device 140 may present the target values of the one or more spectral feature parameters of the target radionuclide on a parameter display region 908. In some embodiments, a plurality of recommendation sets of the target radionuclide are displayed in via a plurality of display sub-regions in the parameter display region 908. For example, as shown in FIG. 9, the parameter display region includes three display sub-regions whose numbers are 1-3 for displaying three recommendation sets.

In some embodiments, the interface 900 includes an adding button 902 and a deleting button 903 for the display sub-regions. The add button 902 is configured to add a new display sub-region to the parameter display region 908. The delete button 903 is configured to remove a display sub-region from the parameter display region 908. In some embodiments, the interface 900 may include a help button 904. The help button 904 is configured to display relevant instructions for registering radionuclide information to assist the user in understanding how to automatically register radionuclide information of a new radionuclide. In some embodiments, the interface 900 may include a scroll button 905. The scroll button 905 is configured to enable horizontal scrolling through the display sub-regions.

In some embodiments, the interface 900 includes a drug carrier list including candidate drug carriers. As shown in FIG. 9, the drug carrier list includes a candidate drug carrier a, a candidate drug carrier b, a candidate drug carrier c, a candidate drug carrier d, a candidate drug carrier e, a candidate drug carrier f, and a candidate drug carrier g. The user may select one or more target drug carriers from the drug carrier list. In response to the selection action, the one or more target drug carriers may be added in to an association drug carrier list.

The interface 900 includes a determination button 906 and a cancel button 907, which are used to confirm and finalize the radionuclide registration or to cancel it, respectively.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lies in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

What is claimed is:

1. A method for registering radionuclide information, implemented on a computing device having at least one processor and at least one storage device, the method comprising:
obtaining a registration instruction for registering a target radionuclide into a radionuclide library of an emission computer tomography (ECT) scanner, the registration instruction including basic information of the target radionuclide;
in response to the registration instruction, obtaining photon energy information of the target radionuclide by controlling the ECT scanner to perform an ECT scan on a subject including the target radionuclide;
obtaining, based on the photon energy information, target values of one or more spectral feature parameters of the target radionuclide; and
registering the basic information and the target values of the one or more spectral feature parameters of the target radionuclide into the radionuclide library of the ECT scanner.

2. The method of claim 1, wherein the one or more spectral feature parameters include at least one of an energy peak, an energy window, a scatter window, or a branching ratio of the target radionuclide.

3. The method of claim 1, wherein the registering the basic information and the target values of the one or more spectral feature parameters of the target radionuclide into the radionuclide library of the ECT scanner comprises:
presenting an energy spectrum curve obtained based on the photon energy information and the target values of the one or more spectral feature parameters to a user; and
in response to a confirmation instruction of the target values, registering the basic information and the target values of the one or more spectral feature parameters of the target radionuclide into the radionuclide library of the ECT scanner.

4. The method of claim 1, further comprising:
presenting a drug carrier list including candidate drug carriers;
obtaining a selection instruction for selecting one or more target drug carriers from the drug carrier list; and
registering associated information between the target radionuclide and the one or more target drug carriers into the radionuclide library.

5. The method of claim 1, wherein the obtaining photon energy information of the target radionuclide comprises:
(a) controlling the ECT scanner to start the ECT scan on the subject;
(b) obtaining current photon energy information collected by the ECT scanner before a current acquisition time;
(c) determining whether a termination condition is satisfied based on the current photon energy information; and
(d) in response to determining that the termination condition is satisfied, designating the current photon energy information as the photon energy information and controlling the ECT scanner to terminate the ECT scan; or in response to determining that the termination condition is not satisfied, determining a next acquisition time, designating the next acquisition time as the current acquisition time, and repeating steps (b) through (d).

6. The method of claim 5, wherein the current acquisition time and the next acquisition time are determined based on a preset acquisition time sequence including a plurality of acquisition times arranged in ascending order.

7. The method of claim 6, wherein the basic information includes a half-life period or a branching ratio of the target radionuclide, the registration instruction further includes an initial activity of the target radionuclide, and the preset acquisition time sequence is determined based on the initial activity and the half-life period or the branching ratio of the target radionuclide.

8. The method of claim 5, wherein the determining whether a termination condition is satisfied based on the current photon energy information comprises:
obtaining previous photon energy information collected by the ECT scanner before a previous acquisition time of the current acquisition time; and
determining whether the termination condition is satisfied based on the previous photon energy information and the current photon energy information.

9. The method of claim 8, wherein the determining whether the termination condition is satisfied based on the previous photon energy information and the current photon energy information comprises:
determining an error factor relating to one or more key energy peaks based on the current photon energy information;
determining a variation factor between the previous photon energy information and the current photon energy information; and
determining whether the termination condition is satisfied based on the error factor and the variation factor.

10. The method of claim 9, wherein in response to determining that the termination condition is not satisfied, the next acquisition time is determined based on the error factor and the variation factor.

11. The method of claim 5, wherein the determining whether a termination condition is satisfied based on the current photon energy information comprises:
presenting an energy spectrum curve corresponding to the current photon energy information to a user; and
in response to a confirmation instruction of the energy spectrum curve, determining that the termination condition is satisfied; or in response to a re-collection instruction, determining that the termination condition is not satisfied.

12. The method of claim 1, wherein the determining, based on the photon energy information, target values of one or more spectral feature parameters of the target radionuclide comprises:
determining, based on the photon energy information, a plurality of recommendation sets, each recommendation set including recommended values of the one or more spectral feature parameters;
for each recommendation set, reconstructing an ECT image of the subject based on ECT data collected by the ECT scanner in the ECT scan and the recommended values in the recommendation set;
presenting the ECT image and the recommended values corresponding to each recommendation set to a user; and
in response to a selection instruction for selecting a target recommendation set from the recommendation sets, designating the recommended values in the target recommendation set as the target values of the one or more spectral feature parameters.

13. The method of claim 1, further comprising:
obtaining a selection instruction for selecting the target radionuclide for a second subject from the radionuclide library;
obtaining third photon energy information collected by the ECT scanner in a second ECT scan on the second subject;
determining whether the selection instruction is accurate based on the target values of the one or more spectral feature parameters and the third photon energy information; and
in response to determining that the selection instruction is not accurate, generating a prompt.

14. The method of claim 1, further comprising:
uploading the basic information and the target values of the one or more spectral feature parameters of the target radionuclide to a shared database.

15. A method for registering radionuclide information, implemented on a computing device having at least one processor and at least one storage device, the method comprising:
controlling an emission computer tomography (ECT) scanner to perform an ECT scan on a subject including a target radionuclide to obtain photon energy information of a target radionuclide;
determining, based on the photon energy information, an energy spectrum curve;
presenting the energy spectrum curve to a user;
obtaining target values of one or more spectral feature parameters of the target radionuclide corresponding to the energy spectrum curve; and
registering the target values of the one or more spectral feature parameters of the target radionuclide into the radionuclide library of the ECT scanner.

16. The method of claim 15, wherein the energy spectrum curve is presented after the ECT scan.

17. The method of claim 15, wherein the energy spectrum curve is presented in real time and varies dynamically during the ECT scan.

18. A method for emission computer tomography (ECT) scan, implemented on a computing device having at least one processor and at least one storage device, the method comprising:
controlling an ECT scanner to perform an ECT scan on a subject including a target radionuclide, wherein the ECT scan includes a first scan period and a second scan period, and the first scan period is earlier than the second scan period;
obtaining, based on scan data collected during the first scan period, target values of one or more spectral feature parameters of the target radionuclide; and
reconstructing an ECT image of the subject based on the target values of one or more spectral feature parameters of the target radionuclide and scan data collected the second scan period.

19. The method of claim 18, wherein the one or more spectral feature parameters include at least one of an energy peak, an energy window, a scatter window, or a branching ratio of the target radionuclide.

20. The method of claim 18, further comprising:
registering the target values of the one or more spectral feature parameters of the target radionuclide into a radionuclide library of the ECT scanner.
